(19) United States
(12) Patent Application Publication
(10) Pub. No.: US 2012/0258269 A1
(43) Pub. Date: Oct. 11, 2012

(54) PREPARING POLYESTER POLYOLS

(75) Inventors: Lionel Gehringer, Schaffhouse-pres-Seltz (FR); Joern Duwenhorst, Lemfoerde (DE); Fin Lammers, Lembruch (DE); Axel Wilms, Frankenthal (DE)

(73) Assignee: BASF SE, Ludwigshafen (DE)

(21) Appl. No.: 13/516,532

(22) PCT Filed: Dec. 15, 2010
(86) PCT No.: PCT/EP2010/069749

§ 371 Date: Jun. 15, 2012

(30) Foreign Application Priority Data
Dec. 16, 2009 (EP) 09179515.3

(51) Int. Cl. C08G 18/32; B32B 1/08; C08G 18/08; C07C 67/08; C07C 69/675

(52) U.S. Cl. 428/36.9; 560/182; 528/59

(57) ABSTRACT

A process for preparing a polyester polyol comprises the steps of:
(a) preparing a reaction mixture comprising the following components:
   A: at least one carboxylic acid recovered from natural raw materials and having at least two acid groups,
   B: at least one polyhydric alcohol,
   C: at least one organic phosphite compound,
   D: at least one Lewis acid;
(b) heating the reaction mixture to a temperature of at least 160° C. and removing the water formed in the course of the reaction;
(c) heating the reaction mixture to a temperature of at least 210° C. under a pressure below 1013 mbar for a period of time in the range from 0.1 to 25 hours.

The present invention relates to a process for preparing polyester polyols, more particularly from natural raw materials, and also to polyester polyols. The present invention further relates to the further conversion of the described polyester polyols to polyurethanes having a light self-color and good mechanical properties.

Polymeric hydroxy compounds such as polyester polyols react with isocyanates to form polyurethanes which have various possible uses, depending on their specific mechanical properties. Polyester polyols in particular have favorable properties and so are used for high-grade polyurethane products. The specific properties of the polyurethanes in question depend substantially on the nature of the polyesterols used.

A particularly important requirement for the production of polyurethanes is that the polyester polyols used have a low acid number (Ullmann's Encyclopedia, Electronic Release, Wiley-VCH-Verlag GmbH, Weinheim, 2000, “Polyesters”, section 2.3 “Quality Specifications and Testing”). The acid number should be low because terminal acid groups react more slowly with diisocyanates than do terminal hydroxyl groups. Polyester polyols having high acid numbers accordingly lead to polyurethanes having a comparatively low molecular weight.

One problem with using polyester polyols having high acid numbers in the manufacture of polyurethanes is that the reaction of the numerous terminal acid groups with isocyanates may result in the formation of an amide bond by elimination of carbon dioxide. The gaseous carbon dioxide can lead to undesirable bubble formation and adverse mechanical properties. Furthermore, free carboxyl groups worsen the catalysis in the polyurethane-forming reaction and also the hydrolysis stability of the polyurethanes produced. This effect can be ameliorated through a higher stabilizer content, but leads to additional costs as well as other undesirable consequences.

There are two types of polyester polyols in terms of chemical structure, viz., the hydroxy carboxylic acid types (AB polyester polyols) and the dihydroxy dicarboxylic acid types (AA-BB polyester polyols).

The former are prepared from just a single monomer by, for example, condensation polymerization of an ω-hydroxy carboxylic acid or by ring-opening polymerization of cyclic esters known as lactones. The AA-BB polyester types are prepared by condensation polymerization of two complementary monomers generally by reacting polyfunctional polyhydroxy compounds (e.g., diols, triols or polyols) with a plurality of functional carboxylic acids, more particularly dicarboxylic acids (e.g., adipic acid or sebacic acid).

The condensation polymerization of polyfunctional polyhydroxy compounds and dicarboxylic acids to form polyester polyols of the AA-BB type on a large industrial scale is generally carried out at high temperatures of 160 to 280° C. This condensation polymerization can be carried out with or without a solvent. One disadvantage of these condensation polymerizations at high temperatures is that they proceed comparatively slowly. To speed the condensation polymerization at high temperatures, esterification catalysts are therefore frequently used. The classic esterification catalysts used here are preferably organometallic compounds, for example titanium tetrabutoxide, tin dioctoate or dibutyltin dilaurate, or acids, for example sulfuric acid, p-toluenesulfonic acid, or bases, for example potassium hydroxide or sodium methoxide. These esterification catalysts are preferably homogeneous and generally remain in the (polyester polyol) product after the reaction has ended.

The use of natural raw materials in the polymer industry is becoming more and more significant since the starting materials are occasionally distinctly cheaper and in some instances available in virtually unlimited volumes.

Natural raw materials are more particularly substances obtained by processing plants or parts of plants (or else animals). Raw materials from renewable resources are characterized by a significant proportion of the carbon isotope ¹⁴C. Its determination allows experimental determination of the proportion of renewable raw materials. Renewable raw materials differ from materials obtained by chemical synthesis and/or by petroleum processing in that they are less homogeneous—their composition can vary to a distinctly greater extent.

These fluctuations in the composition of natural raw materials and the presence of further, difficult-to-remove concomitants, such as degradation products or impurities, frequently lead to problems in further processing and therefore limit the industrial use of these materials.

Fluctuations in the composition of natural raw materials are for example dependent on factors such as the climate and region in which the plant grows, the time of year at which it is harvested, variations between biological species and subspecies and the type of extraction method used to recover the natural raw material (extrusion, centrifugation, filtering, distillation, cutting, pressing, etc.).

Preparing polyester polyols by reaction of starting materials recovered from natural raw materials is of enormous interest specifically for the production of (thermoplastic) polyurethanes for the shoe industry for example. Owing to the impurities and/or degradation products which may be present in feed stocks obtained from natural raw materials, polyester polyols prepared therefrom have hitherto not found any large scale industrial use. One reason for this are the substantial discoloration of the recovered polyester polyols which results from the impurities and/or defects in the functionality. This substantial discoloration means that no industrially sensible conversion of these polyester polyols into polyurethanes is possible. The products are often so dark that they cannot be used for demanding optical applications. Technical grade fluids, such as liquid polyester polyols, frequently have an undesirable yellowness due to impurities or degradation products in some instances.

Use in thermoplastic polyurethanes (TPUs) requires maintenance of a polyester polyol functionality of two (2) as a precondition for good processibility in injection molding and more particularly in extrusion molding. Even very small amounts of higher-functional impurities can lead to disadvantageous crosslinking in the thermoplastic polyurethane.

Technical grade fluids can be color classified according to the APHA/HAZEN color assessment scheme. Its recommendation by the American Public Health Administration (APHA) led to its name.

The principle of this color assessment scheme is the visual comparison of analytical samples in standardized vessels with yellow standard solutions graduated in concentration. The APHA-/HAZEN color number utilizes an acidic solution of potassium hexachloroplatinate(IV) and cobalt(II) chloride in accordance with an 1892 proposal by Allen Hazen. Comparator solutions are then assigned a color number in accordance with their platinum content in mg/l (range is 0-600).

WO 1992/00947 describes processes for esterifying oxyhydrocarbon polyols by adding reducing agents, for example sodium borohydride, lithium aluminum hydride and sodium, which lead to a lighter color on the part of the product. The synthesis for preparing fatty acid esters of some alkylglucosides and also the transesterification and cyclization from fatty acid esters onto lower alcohols also is described. The resulting polyol mixtures, which tend to darken over time, are treated with the reducing agent in the process described before and during the esterifying step. An additional step prior to the esterification comprises for example performing a cyclization of sorbitol to sorbitan at 170° C. in the presence of hypophosphite ions. The amount of hypophosphite ions used is specified as 0.2% to 0.7% by weight based on the polyol component.

EP-A 0 572 256 describes preparing biodegradable high molecular weight aliphatic polyesters. For example, the molten aliphatic polyester has added to it a phosphorus component which may be selected from the group consisting of organic phosphoric esters, such as triphenyl phosphite, diphenyl isodecyl phosphite, phenyl diisodecyl phosphite, tris(mono- and/or dinonylphenyl) phosphite and trisisodecyl phosphite. This phosphorus-containing component is further stated to act as a stabilizer that enhances thermal stability, prevents discoloration and avoids viscosity fluctuations.

U.S. Pat. No. 4,677,154 describes preparing discoloration-eliminated thermoplastic polyurethanes. The specific production process and the addition of a specific stabilizer package (BHT) consisting of various components, including phosphites, make it possible to produce reaction products in the form of less colored or light-colored thermoplastic polyurethanes.

EP-A 1 195 395 describes thermoplastically processible polyurethane elastomers of improved self-color. The use of specifically substituted pentaerythritol diphosphites makes it possible to achieve an improved self-color. The pentaerythritol diphosphite is added before or during polyurethane production.

DE-A 10 121 866 describes a process for producing light-colored fatty acid polyol esters by reaction of fatty acid alkyl esters with polyols. The reaction is carried out in the presence of reducing agents and alkali metal bases.

JP-A 7309 937 describes low-colored polyesters and their production. The production process utilizes various stabilizers including tris(2,4-di-t-butylphenyl) phosphites.

WO 2008/031592 presents a process for preparing dianhydrohexitol-based polyesters. The process utilizes succinic acid, glutaric acid, adipic acid or sebacic acid among other dicarboxylic acids. Preferred alcohols are 1,3-propanediol, 1,4-butanediol, 2,3-butanediol and/or trimethylolpropane.

None of the processes from the cited prior art is based on natural raw materials leading without further purification to light-colored polyester polyols which are then suitable for conversion into polyurethanes.

It is an object of the present invention to provide a process whereby natural raw materials, more particularly natural carboxylic acids and/or polyols, can be used to prepare polyester polyols that have minimal coloration and more particularly positively influence the further reaction to form polyurethanes.

We have found that this object is achieved, surprisingly, by providing a process for preparing polyester polyols wherein organic phosphites are added to at least dicarboxylic acids recovered from natural materials and light-colored polyester polyols are obtained. These polyester polyols can then be converted into polyurethanes of minimal (light) self-color.

The thermoplastic polyurethane is also notable for high transparency.

The present invention accordingly provides a process for preparing a polyester polyol comprising the steps of:
(a) preparing a reaction mixture comprising the following components:
   A: at least one carboxylic acid recovered from natural raw materials and having at least two acid groups,
   B: at least one polyhydric alcohol,
   C: at least one organic phosphite compound,
   D: at least one Lewis acid;
(b) heating the reaction mixture to a temperature of at least 160° C. and removing the water formed in the course of the reaction;
(c) heating the reaction mixture to a temperature of at least 210° C. under a pressure below 1013 mbar for a period of time in the range from 0.1 to 25 hours.

The duration of the vacuum phase (step c) is frequently in the range from 1 to 22 hours and preferably in the range from 5 to 20 hours.

The organic carboxylic acids which have at least two acid groups (carboxyl groups) are recoverable from natural raw materials by specific processing methods. For instance, treating castor oil with sodium hydroxide or potassium hydroxide at high temperatures in the presence of comparatively long-chain alcohols (such as 1- or 2-octanol) will result in sebacic acid being obtainable as an important raw material in a purity of >99.5% among other products according to reaction conditions. Sebacic acid (1,8-octanedicarboxylic acid) is a member of the homologous series of aliphatic dicarboxylic acids.

Succinic acid and/or 2-methylsuccinic acid are particularly suitable as well as sebacic acid. They are obtainable from natural raw materials such as sugar or corn (maize), by fermentation.

Component A in the process of the present invention may comprise more particularly one or more, for example two or three, different carboxylic acids from the group of C₂ to C₁₂ dicarboxylic acids. By C₂ to C₁₂ dicarboxylic acids are meant dicarboxylic acids which are aliphatic or branched and have two to twelve carbon atoms. It is also possible for component A to comprise C₂ to C₁₄ dicarboxylic acids, preferably C₄ to C₁₂ dicarboxylic acids and more preferably C₆ to C₁₀ dicarboxylic acids.

The at least one dicarboxylic acid recovered from natural raw materials may further also be present as a carboxylic diester or as a carboxylic anhydride.

Dicarboxylic acid (A) may in principle comprise aliphatic and/or aromatic dicarboxylic acids. In one particularly preferred embodiment of the present invention, the dicarboxylic acid (A) recovered from natural raw materials is selected from the group consisting of sebacic acid, azelaic acid, dodecanedioic acid and succinic acid. The polyhydric alcohol (B) in the process of the present invention is more particularly selected from the group consisting of 1,3-propanediol, 1,2-ethanediol and butanediols (particularly 1,4-butanediol). In a further preferred embodiment of the present invention, component A comprises sebacic acid recovered from renewable raw materials.

In one embodiment of the present invention, component B is an aliphatic C₂ to C₆ diol. Useful aliphatic C₂ to C₆ diols include, in particular, polyhydric alcohols (B), preferably diols component such as, for example, ethylene glycol, diethylene glycol, 3-oxapentane-1,5-diol, 1,3-propanediol, 1,2-propanediol, dipropylene glycol, 1,4-butanediol, 1,5-pentanediol, 1,6-hexanediol, 2-methyl-1,3-propanediol and 3-methyl-1,5-pentanediol.

A further embodiment utilizes an aliphatic diol having 2 to 14 carbon atoms and more particularly a C₄ to C₁₂ diol as component B.

Alcohols having three or more OH groups can also be used to enhance the functionality of the polyester alcohols. Examples of alcohols having three or more OH groups are glycerol, trimethylolpropane and pentaerythritol. It is also possible to use oligomeric or polymeric products having two or more hydroxyl groups. Examples thereof are polytetrahydrofuran, polylactones, polyglycerol, polyetherols, polyesterol or α,ω-dihydroxypolybutadiene.

1,3-Propanediol may comprise synthetically produced 1,3-propanediol, but in particular 1,3-propanediol from renewable raw materials (“biobased 1,3-propanediol”). Biobased 1,3-propanediol is obtainable from maize (corn) and/or sugar for example. A further possibility is the conversion of waste glycerol from biodiesel production. In one particularly preferred embodiment of the present invention, component B comprises 1,3-propanediol, with this 1,3-propanediol preferably also being recovered from renewable raw materials.

The process of the present invention can utilize any organic phosphite compound (C) known to a person skilled in the art. Preference is given to using organic phosphite compounds of the type POR₃, where R may be a linear, branched and/or aromatic C₁ to C₁₂ radical. Organic phosphites are esters of phosphonic acids. Examples of commercially available organic phosphites are the products of the Irgafos® range from Ciba Speciality Chemicals (Switzerland) or BASF SE (Germany, Ludwigshafen).

In one particularly preferred embodiment of the present invention, component C comprises at least one organic phosphite compound selected from the group consisting of bis(2,4-di-tert-butyl-6-methylphenyl)ethyl phosphite, tris(nonylphenyl)phosphite or the reaction product of phosphorus trichloride with 1,1′-biphenyl and 2,4-bis(tert-butyl)phenol (Irgaphos® P-EPQ).

The phosphite compound may be used in a concentration of 100 to 10 000 ppm, particularly 200 to 2000 ppm and preferably in the range from 500 to 1000 ppm (based on the total amount of stabilizer). The phosphite compound is preferably used in a concentration of 5 to 1500 ppm, particularly 10 to 400 ppm and more preferably 20 to 150 ppm, based on the active sites. Active sites are the chemical sites that prevent a color reaction. In this case, the active sites are the phosphorus atoms of the phosphites.

The process of the present invention may utilize the Lewis acids known to a person skilled in the art. Lewis acids are electron pair acceptors in that they are capable of accepting an electron pair to form a covalent bond. Known examples of Lewis acids are BF₃, AlH₃, SiF₄, PF₃, SnCl₄, SO²⁺, SO³⁺, H⁺, Mg²⁺, Al³⁺, Cu²⁺, Hg⁺, Ti⁴⁺ and Sn²⁺.

In one preferred embodiment of the present invention, the at least one Lewis acid is selected from the group consisting of titanium tetrabutoxide, titanium tetraisopropoxide, tin dioctoate, dibutyltin laurate and tin chlorides.

In a particular embodiment of the present invention, the preparing of the reaction mixture in step (a) is effected by first mixing components A, B and D and only then adding component C. Component C can in principle be added to the reaction mixture at any time prior to the start of the reaction of the dicarboxylic acid to form the polyester polyol, generally the addition takes place at temperatures of 20° C. to not more than 120° C.

The process of the present invention is preferably carried out without a solvent.

The process of the present invention provides more particularly polyester polyols having a low APHA/HAZEN color number. After the process of the present invention has been carried out, the polyester polyol may preferably have a color number between 10 and 200 APHA/HAZEN. APHA/HAZEN color numbers between 10 and 195, in particular between 10 and 150, and particulary below 150 are preferred.

The acid numbers of the polyester polyols obtained are preferably in the region of less than 3 g KOH/kg, preferably in the region of less than 2 g KOH/kg and more particularly in the region of less than 1 g KOH/kg. The acid number is used to determine the level of free organic acids in the polyester polyol. The acid number is determined for example by the amount of KOH in mg (or g of KOH) needed to neutralize an amount of 1 g (or 1 kg, respectively) of the sample.

The customary apparatus for preparing polyester polyols is known to a person skilled in the art.

The present invention further comprises a polyester polyol product obtainable by the process of the present invention.

A preferred embodiment of the present invention provides polyester polyols obtainable by the above-described process utilizing sebacic acid as component A.

The present invention further provides a process for preparing a thermoplastic polyurethane by reacting a polyester polyol obtained (or obtainable) according to the process of the present invention with one or more organic diisocyanates (or polyisocyanates).

Polyurethanes can in principle be prepared according to known processes, batchwise or continuously, for example using reactive extruders or the belt process according to one-shot processes or the prepolymer process (including multi-stage prepolymer processes, see U.S. Pat. No. 6,790,916 for example), but preferably according to the one-shot process. In these processes, the reaction components—polyesterol, chain extender, isocyanate (see Table 1) and optionally auxiliaries and additives (more particularly UV stabilizers)—can be mixed with one another in succession or simultaneously, and the reaction ensues immediately.

Further information concerning the abovementioned auxiliary and additive materials is derivable from the technical literature, for example from “Plastics Additive Handbook”, 5th Edition, H. Zweifel, ed, Hanser Publishers, Munich, 2001; H. Saunders and K. C. Frisch “High Polymers”, Volume XVI, Polyurethane, Parts 1 and 2, Verlag Interscience Publishers 1962 and 1964; Taschenbuch fur Kunststoff-Additive by R. Gachter and H. Muller (Hanser Verlag Munich 1990) or DE-A 29 01 774.

Apparatus for preparing polyurethanes is known to a person skilled in the art.

The polyurethane obtained from a polyester polyol obtained according to the process of the present invention is a thermoplastic polyurethane in particular. Thermoplastic polyurethanes are hereinafter also referred to as TPUs.

The present invention further provides for the use of a polyester polyol obtained according to the process of the present invention in the manufacture of polyurethanes (hereinafter also referred to as Pus), more particularly PU flexible foam, PU rigid foam, polyisocyanurate (PIR) rigid foam, noncellular PU materials or polyurethane dispersions. The polyurethanes described above are useful inter alia in the manufacture of mattresses, shoe soles, gaskets, hoses, flooring, profiles, coatings, adhesives, sealants, skis, auto seats, running tracks in stadia, dashboards, various moldings, potting compounds, self-supporting film/sheet, fibers, nonwovens and/or cast floors.

The thermoplastic polyurethanes obtained according to the process of the present invention can be transparent and have a yellow index (YI) of less than 20. The yellow index refers generally to a parameter involved in the measurement of the color of transparent plastics.

The use of polyester polyols in the manufacture of polyurethanes further relates to the manufacture of (foamed) flexible foam and/or compact casting systems.

The present invention further provides for the use of a thermoplastic polyurethane obtained according to the process of the present invention in the manufacture of moldings, hoses, self-supporting film/sheet and/or fibers.

The present invention further relates to a molding, a self-supporting film/sheet, a hose or a fiber obtained from a thermoplastic polyurethane based on the process of the present invention.

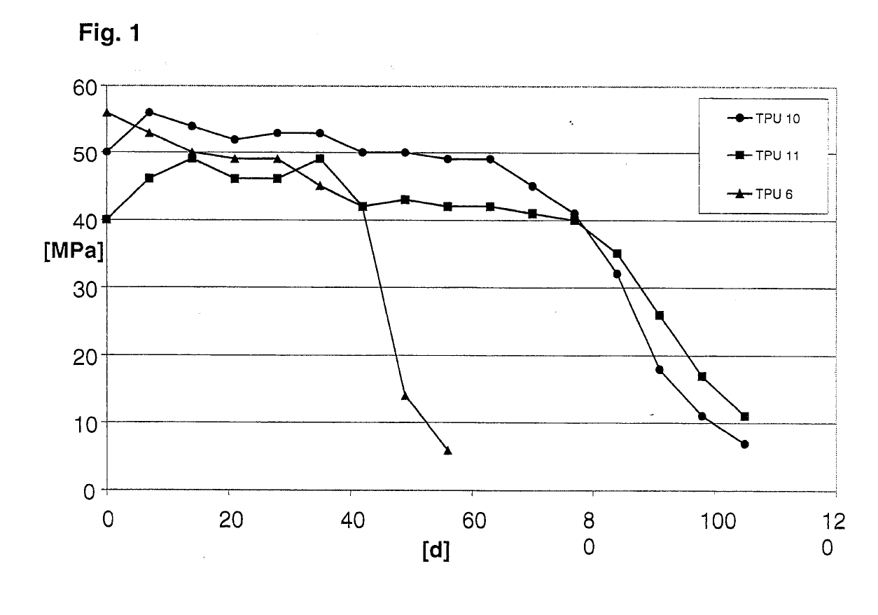

FIG. 1 shows a diagram concerning the mechanical properties of the thermoplastic polyurethanes as per the examples featuring thermoplastic polyurethane [TPU] numbers 6, 10 and 11. The diagram shows the dependence of tensile strength [MPa] on days [d] immersion in hot water at 80° C.

The illustration shows that use of organic phosphites in the process does not lead to loss of water resistance on the part of the product.

### EXAMPLES

Color number was determined using an LICO150 color number measuring instrument from Hach Lange GmbH. Before being introduced into a disposable round glass cuvette (11 mm in diameter), the samples were heated to 90° C. in a thermal cabinet and then introduced into the cuvette without bubbles (with the aid of an ultrasonic bath). The result of the color determination can be reported as iodine color number and/or as Hazen color number (APHA).

### Example 1 (Comparative Example)

4754.2 g of sebacic acid, 2092.9 g of 1,3-propanediol, 1 ppm of titanium tetrabutoxide and 5 ppm of tin octoate were introduced at room temperature into a round flask having a capacity of 12 liters. The mixture was gradually heated to 180° C. with stirring and then left at 180° C. for 3 hours with stirring. In the process, the resulting water was removed by distillation at atmospheric pressure.

Thereafter, the mixture was heated to 220° C. in vacuo and left at 220° C. under a vacuum of 40 mbar until an acid number of less than 1 mg KOH/g was reached. The resulting liquid polyester polyol had the following characteristic values:
hydroxyl number: 81.0 mg KOH/g
acid number: 0.1 mg KOH/g
water: 0.002 (% by weight)
viscosity: 305 mPa·s (at 75° C.)
color number: 422 APHA/Hazen

### Example 2 (Comparative Example)

4754.2 g of sebacic acid, 2092.9 g of biobased 1,3-propanediol (from DuPont), 1 ppm of titanium tetrabutoxide and 5 ppm of tin octoate were introduced at room temperature into a round flask having a capacity of 12 liters. The mixture was gradually heated to 180° C. with stirring and then left at 180° C. for 3 hours with stirring. In the process, the resulting water was removed by distillation at atmospheric pressure.

Thereafter, the mixture was heated to 220° C. in vacuo and left at 220° C. under a vacuum of 40 mbar until an acid number of less than 1 mg KOH/g was reached. The resulting liquid polyester polyol had the following characteristic values:
hydroxyl number: 74.5 mg KOH/g
acid number: 0.1 mg KOH/g
water: 0.003 (% by weight)
viscosity: 390 mPa·s (at 75° C.)
color number: 600 APHA/Hazen

### Example 3 (Comparative Example)

4627.6 g of sebacic acid, 2198.0 g of 1,3-propanediol, 1 ppm of titanium tetrabutoxide and 5 ppm of tin octoate were introduced at room temperature into a round flask having a capacity of 12 liters. The mixture was gradually heated to 180° C. with stirring and then left at 180° C. for 3 hours with stirring. In the process, the resulting water was removed by distillation at atmospheric pressure.

Thereafter, the mixture was heated to 220° C. in vacuo and left at 220° C. under a vacuum of 40 mbar until an acid number of less than 1 mg KOH/g was reached. The resulting liquid polyester polyol had the following characteristic values:
hydroxyl number: 112.0 mg KOH/g
acid number: 0.04 mg KOH/g
water: 0.004 (% by weight)
viscosity: 175 mPa·s (at 75° C.)
color number: 380 APHA/Hazen

### Example 4

4754.2 g of sebacic acid, 2092.9 g of 1,3-propanediol, 160 ppm of Irgafos 38 (from Ciba), 1 ppm of titanium tetrabutoxide and 5 ppm of tin octoate were introduced at room temperature into a round flask having a capacity of 12 liters. The mixture was gradually heated to 180° C. with stirring and then left at 180° C. for 3 hours with stirring. In the process, the resulting water was removed by distillation at atmospheric pressure.

Thereafter, the mixture was heated to 220° C. in vacuo and left at 220° C. under a vacuum of 40 mbar until an acid number of less than 1 mg KOH/g was reached. The resulting liquid polyester polyol had the following characteristic values:
hydroxyl number: 79.2 mg KOH/g
acid number: 0.7 mg KOH/g
water: 0.003 (% by weight)
viscosity: 370 mPa·s (at 75° C.)
color number: 260 APHA/Hazen

### Example 5

4754.2 g of sebacic acid, 2092.9 g of 1,3-propanediol, 160 ppm of Irgafos 38 (from Ciba), 1 ppm of titanium tetrabutoxide and 5 ppm of tin octoate were introduced at room temperature into a round flask having a capacity of 12 liters. The mixture was gradually heated to 180° C. with stirring and then left at 180° C. for 3 hours with stirring. In the process, the resulting water was removed by distillation at atmospheric pressure.

Thereafter, the mixture was heated to 220° C. in vacuo and left at 220° C. under a vacuum of 40 mbar until an acid number of less than 1 mg KOH/g was reached. The resulting liquid polyester polyol had the following characteristic values:
hydroxyl number: 73.0 mg KOH/g
acid number: 0.6 mg KOH/g
water: 0.004 (% by weight)
viscosity: 260 mPa·s (at 75° C.)
color number: 195 APHA/Hazen

### Example 6

4754.2 g of sebacic acid, 2092.9 g of 1,3-propanediol, 8000 ppm of Irgafos 38 (from Ciba), 1 ppm of titanium tetrabutoxide and 5 ppm of tin octoate were introduced at room temperature into a round flask having a capacity of 12 liters. The mixture was gradually heated to 180° C. with stirring and then left at 180° C. for 3 hours with stirring. In the process, the resulting water was removed by distillation at atmospheric pressure.

Thereafter, the mixture was heated to 220° C. in vacuo and left at 220° C. under a vacuum of 40 mbar until an acid number of less than 1 mg KOH/g was reached. The resulting liquid polyester polyol had the following characteristic values:
hydroxyl number: 73.7 mg KOH/g
acid number: 0.1 mg KOH/g
water: 0.002 (% by weight)
viscosity: 380 mPa·s (at 75° C.)
color number: 135 APHA/Hazen

### Example 7

4754.2 g of sebacic acid, 2092.9 g of 1,3-propanediol, 2200 ppm of Irgafos TNPP (from Ciba), 1 ppm of titanium tetrabutoxide and 5 ppm of tin octoate were introduced at room temperature into a round flask having a capacity of 12 liters. The mixture was gradually heated to 180° C. with stirring and then left at 180° C. for 3 hours with stirring. In the process, the resulting water was removed by distillation at atmospheric pressure.

Thereafter, the mixture was heated to 220° C. in vacuo and left at 220° C. under a vacuum of 40 mbar until an acid number of less than 1 mg KOH/g was reached. The resulting liquid polyester polyol had the following characteristic values:
hydroxyl number: 77.4 mg KOH/g
acid number: 0.55 mg KOH/g
water: 0.002 (% by weight)
viscosity: 380 mPa·s (at 75° C.)
color number: 150 APHA/Hazen

### Example 8

4754.2 g of sebacic acid, 2092.9 g of biobased 1,3-propanediol (from DuPont), 800 ppm of Irgafos TNPP (from Ciba), 1 ppm of titanium tetrabutoxide and 5 ppm of tin octoate were introduced at room temperature into a round flask having a capacity of 12 liters. The mixture was gradually heated to 180° C. with stirring and then left at 180° C. for 3 hours with stirring. In the process, the resulting water was removed by distillation at atmospheric pressure.

Thereafter, the mixture was heated to 220° C. in vacuo and left at 220° C. under a vacuum of 40 mbar until an acid number of less than 1 mg KOH/g was reached. The resulting liquid polyester polyol had the following characteristic values:
hydroxyl number: 78.6 mg KOH/g
acid number: mg KOH/g
water: 0.60 (% by weight)
viscosity: 370 mPa·s (at 75° C.)
color number: 190 APHA/Hazen

### Example 9

4627.6 g of sebacic acid, 2198.0 g of 1,3-propanediol, 800 ppm of Irgafos 38 (from Ciba), 1 ppm of titanium tetrabutoxide and 5 ppm of tin octoate were introduced at room temperature into a round flask having a capacity of 12 liters. The mixture was gradually heated to 180° C. with stirring and then left at 180° C. for 3 hours with stirring. In the process, the resulting water was removed by distillation at atmospheric pressure.

Thereafter, the mixture was heated to 220° C. in vacuo and left at 220° C. under a vacuum of 40 mbar until an acid number of less than 1 mg KOH/g was reached. The resulting liquid polyester polyol had the following characteristic values:
hydroxyl number: 115.6 mg KOH/g
acid number: 0.37 mg KOH/g
water: 0.003 (% by weight)
viscosity: 200 mPa·s (at 75° C.)
color number: 128 APHA/Hazen

### Example 10

4627.6 g of sebacic acid, 2198.0 g of 1,3-propanediol, 800 ppm of Irgafos P-EPQ (from Ciba), 1 ppm of titanium tetrabutoxide and 5 ppm of tin octoate
hydroxyl number: 116.1 mg KOH/g
acid number: 0.10 mg KOH/g
water: 0.005 (% by weight)
viscosity: 182 mPa·s at 75° C.
color number: 237 APHA/Hazen

General protocol for preparing thermoplastic polyurethanes (TPUs)

In a 2 liter tinplate bucket, the Table 2 amount of polyol from the appropriate inventive or comparative example was admixed with the additives KV1 and also S1-S3 reported in Table 2 by addition to the hot polyester polyol at 80° C. After subsequent heating of the mixture to 80° C., MDI (4,4-methyl diisocyanate) was added as per Table 2, followed by stirring of the mixture until the temperature of the exothermic reaction had risen to 110° C. The reaction mixture was subsequently poured into a shallow dish and heat conditioned at 125° C. on a hotplate for 10 minutes. Thereafter, the resulting hide was heat conditioned at 80° C. in a heating cabinet for 15 h. The hide was then granulated and made into 2 mm and 6 mm test plaques in accordance with general processing methods for TPU.

TABLE 1
Starting materials used for polyurethanes

| | Product | |
|---|---|---|
| Designation | number | Chemical composition |
| Polyol | Polyester polyol | Obtained as per preceding examples or |
| | | commercial polyols: butyl adipates, |
| | | molecular weight: 1000 g/mol, |
| | | functionalities: 2 |
| KV1 | Chain extender | 1,4-Butanediol |
| Isocyanate | MDI | Diphenylmethane diisocyanate |
| S1 | Hydrolysis | Polymeric carbodiimide |
| | stabilizer | |
| S2 | Antioxidant 1 | Tetrakis[methylene (3,5-di-tert-butyl- |
| | | 4-hydroxy-hydrocinnamate)]methane |
| S3 | Antioxidant 2 | Tris(nonylphenyl) phosphite |

TABLE 2
Overview of composition of TPUs (hand casts)

| | Polyester polyol | | | | | | |
|---|---|---|---|---|---|---|---|
| | as per preceding | | | | | | |
| | examples | | | | S1 | S2 | S3 |
| | Type | [g] | KV [g] | MDI [g] | [g] | [g] | [g] |
| TPU 2¹ | Comparative | 1000 | 232.92 | 816.88 | 8.0 | — | — |
| | example 2 | | | | | | |
| TPU 3¹ | Example 6 | 1000 | 232.58 | 814.21 | 8.0 | — | --- |
| TPU 4¹ | Example 7 | 1000 | 234.20 | 826.83 | 8.0 | 8.00 | — |
| TPU 5¹ | Example 8 | 1000 | 234.74 | 832.04 | 10.00 | 7.40 | — |
| TPU 6² | Purchased | 1000 | 149.04 | 670.00 | 8.00 | — | — |
| | polyol | | | | | | |
| TPU 7² | Purchased | 1000 | 149.04 | 670.00 | 8.00 | 7.78 | — |
| | polyol | | | | | | |
| TPU 8 | Example 3 | 700 | 174.73 | 662.80 | 5.60 | 7.78 | 0.00 |
| TPU 9 | Example 3 | 700 | 174.73 | 662.80 | 5.60 | 7.84 | 0.78 |
| TPU 10¹ | Example 9 | 700 | 175.86 | 671.54 | 5.60 | 7.84 | 0.78 |
| TPU 11¹ | Example 10 | 700 | 176.01 | 672.75 | 5.60 | 7.84 | 0.78 |

¹raw material from renewable resources
²purchased polyol: butyl adipates, molecular weight: 1000 g/mol, functionalities: 2 as per Table 1

TABLE 3
Mechanical properties of polyurethanes

| | | | | | | | | YI |
|---|---|---|---|---|---|---|---|---|
| | | | | Tongue | | | MFR | (Yellow |
| | | Tensile | Breaking | tear | | | (melt flow | Index, |
| | Hardness | strength | extension | resistance | Abrasion | Density | rate) | unconditioned) |
| | [Shore D] | [MPa] | [%] | [N/mm] | [mm³] | [g/cm³] | [g/10 min] | [ ] |
| Measured | DIN | DIN | DIN | DIN ISO | DIN ISO | DIN EN | DIN EN | ASTM |
| to standard | 53505 | 53504 | 53504 | 34-1, B (b) | 4649 | ISO 1183-1, A | ISO 1133 | E313 |
| TPU 2 | 50 | 34 | 470 | 114 | 86 | 1.189 | 43.3 | 29 |
| | | | | | | | (200° C./ | |
| | | | | | | | 21.6 kg) | |
| TPU 3 | 58 | 49 | 420 | 135 | 36 | 1.188 | 29.6 | 6.0 |
| | | | | | | | (200° C./ | |
| | | | | | | | 21.6 kg) | |
| TPU 4 | 49 | 52 | 420 | 132 | 26 | 1.188 | 15.4 | 8.2 |
| | | | | | | | (210° C./ | |
| | | | | | | | 21.6 kg) | |
| TPU 5 | 52 | 53 | 470 | 138 | 28 | 52 | 35.8 | 19 |
| | | | | | | | (210° C./10 kg) | |
| TPU 6 | 51 | 68 | 480 | 109 | 35 | 1.216 | 35 | 5.8 |
| | | | | | | | (190° C./21.6 kg) | |
| TPU 7 | 51 | 57 | 480 | 110 | 26 | 1.216 | 35.1 | 1.2 |
| | | | | | | | (190° C./21.6 kg) | |
| TPU 8 | 51 | 26 | 470 | 117 | 120 | 1.199 | 77.5 | 14.0 |
| | | | | | | | (210° C./2.16 kg) | |
| TPU 9 | 50 | 27 | 450 | 126 | 114 | 1.198 | 63.0 | 16.0 |
| | | | | | | | (210° C./2.16 kg) | |
| TPU 10 | 64 | 50 | 460 | 171 | 37 | 1.2 | 37.7 | 11.4 |
| | | | | | | | (230° C./2.16 kg) | |
| TPU 11 | 63 | 40 | 430 | 166 | 44 | 1.2 | 30.6 | 13.1 |
| | | | | | | | (230° C./2.16 kg) | |

Hardness, tensile strength, breaking extension, tongue tear resistance, abrasion and density were each measured to the particular DIN standard indicated.

There follows 1 sheet of drawings.

1. A process for preparing a polyester polyol, the process comprising of:
(a) preparing a reaction mixture comprising:
A: at least one carboxylic acid recovered from a natural raw material and having at least two acid groups, selected from the group consisting of sebacic acid, azelaic acid, dodecanedioic acid and succinic acid,
B: at least one polyhydric alcohol,
C: at least one organic phosphite compound,
D: at least one Lewis acid;
(b) heating the reaction mixture to a temperature of at least 160° C. and removing water formed during a reaction; and
(c) heating the reaction mixture to a temperature of at least 210° C. at a pressure below 1013 mbar for 0.1 to 25 hours,
to obtain a polyester polyol.

2. The process of claim 1 wherein (a) comprises first mixing A, B and D, and then adding C.

3. (canceled)

4. The process of claim 1, wherein A comprises sebacic acid recovered from a renewable raw material.

5. The process of claim 1, wherein B comprises an aliphatic C₂ to C₆ diol.

6. The process of claim 1, wherein B comprises 1,3-propanediol or 1,4-butanediol.

7. The process of claim 1, wherein C comprises at least one organic phosphite compound selected from the group consisting of bis(2,4-di-tert-butyl-6-methylphenyl)ethyl phosphite, tris(nonylphenyl)phosphite or a reaction product of phosphorus trichloride with 1,1′-biphenyl and 2,4-bis(tert-butyl)phenol.

8. The polyester polyol obtained by the process of claim 1.

9. The polyester polyol of claim 8 wherein A comprises sebacic acid recovered from a renewable raw material, and B comprises a diol.

10. A process for preparing a polyurethane by reacting the polyester polyol obtained by the process claim 1 with one or more organic diisocyanates.

11. A thermoplastic polyurethane obtained by the process of claim 10.

12. (canceled)

13. A process of manufacturing a molding, a hose, a self-supporting film/sheet, or a fiber, the process comprising the process of claim 10.

14. A molding, a self-supporting film/sheet, a hose or a fiber comprising the thermoplastic polyurethane of claim 11.

15. The process of claim 1, wherein (c) is performed for 5 to 20 hours.

16. The process of claim 1, wherein B comprises 1,3-propanediol.

17. The process of claim 1, wherein B comprises 1,4-butanediol.

18. The process of claim 1, wherein D is selected from the group consisting of titanium tetrabutoxide, titanium tetraisopropoxide, tin dioctoate, dibutyltin laurate, and tin chloride.

19. The process of claim 1, wherein the polyester polyol obtained has a color number between 10 and 200 APHA/HAZEN.

20. The process of claim 1, wherein the polyester polyol obtained has an acid number less than 3 g KOH/kg.

21. The process of claim 1, wherein the polyester polyol obtained has an acid number less than 1 g KOH/kg.

22. The process of claim 1, performed without a solvent.
